(19) United States
(12) Patent Application Publication
(10) Pub. No.: US 2024/0282440 A1
(43) Pub. Date: Aug. 22, 2024

(54) APPARATUS AND METHOD FOR DETERMINING RARITY OF BIOMETRIC INFORMATION SIGNAL

(71) Applicant: Xandar Kardian, Inc., New York, NY (US)

(72) Inventors: Jeong Woo Choi, Seoul (KR); Linda Susanna Mayhue, Prince Albert (CA); Sun Jong Yang, Gangwon-Do (KR); William Richard Marritt, Riverside, IL (US)

(73) Assignee: Xandar Kardian, Inc., New York, NY (US)

(21) Appl. No.: 18/372,362

(22) Filed: Sep. 25, 2023

(30) Foreign Application Priority Data
Feb. 22, 2023 (KR) 10-2023-0023602

(51) Int. Cl. G16H 40/63

(52) U.S. Cl. CPC G16H 40/63

(57) ABSTRACT

The present invention provides a method of determining rarity of a signal. The method of determining rarity of a signal includes receiving group biometric information data for a certain period through a sensor, extracting, by an extraction processor, partial data in the received group biometric information data, calculating, by a comparison processor, a cumulative distribution function (CDF) based on the group biometric information data, calculating, by the comparison processor, a sample rarity score of the extracted partial data based on the calculated CDF, and calculating a group rarity score from the sample rarity score, and comparing, by the comparison processor, the group rarity score of the extracted partial data with a threshold value to determine whether a singularity is present.

### CROSS-REFERENCE TO RELATED APPLICATION

This application claims, under 35 U.S.C. § 119(a), the benefit of and priority to Korean Patent Application No. 10-2023-0023602 filed on Feb. 22, 2023, the entire contents of which are incorporated herein by reference.

### BACKGROUND

### (a) Technical Field

The present disclosure relates to an apparatus and method for determining rarity of a biometric information signal. More particularly, it relates to an apparatus and method for determining rarity of a biometric information signal for determining whether a singularity has occurred by determining rarity of partial data extracted from a corresponding group based on group biometric information data measured for a certain period.

### (b) Background Art

With development of medicine and extension of life expectancy, interest in medical devices has been increasing, and a range thereof has been expanding to small medical devices and health care devices that may be owned or carried by individuals in addition to medium and large medical devices used in hospitals and inspection institutions. Medical devices that measure biometric information may be broadly classified into invasive devices and noninvasive devices. A noninvasive device has an advantage of being able to relatively simply detect biometric information without causing pain to a subject.

Moreover, in the case of technology for measuring biometric information using a noninvasive sensor, when corresponding data is analyzed using one piece of measurement data, analysis which is inconsistent in terms of information consistency of a subject to be measured is provided, and thus various studies have been conducted on analysis methods to maintain consistency.
Patent Document 1: Korean Patent Publication No. 10-20220115734

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention has been made in an effort to solve the above-described problems associated with prior art. An object of the present invention is to provide an apparatus and method for determining rarity of a biometric information signal capable of analyzing rarity of partial data extracted based on data measured for a predetermined time rather than analyzing one piece of individual data.

In addition, the present invention provides an apparatus and method for determining rarity of a biometric information signal capable of determining whether a singularity is present by calculating rarity of partial data extracted based on a cumulative distribution function calculated through data measured for a predetermined time and comparing the rarity with data measured over the same period.

The objects of the present invention are not limited to the above-mentioned objects, and other objects of the present invention not mentioned above may be understood by the following description and may be more clearly understood by the examples of the present invention. In addition, the objects of the present invention may be realized by the means and combinations indicated in the claims.

A method of determining rarity of a biometric information signal for achieving the objects of the present invention described above includes the following configuration.

In one aspect, the present invention provides a method of determining rarity of a biometric information signal including receiving group biometric information data for a certain period through a sensor, extracting, by an extraction processor, partial data in the received group biometric information data, calculating, by a comparison processor, a cumulative distribution function (CDF) based on the group biometric information data, calculating, by the comparison processor, a sample rarity score of the extracted partial data based on the calculated CDF, and calculating a group rarity score from the sample rarity score, and comparing, by the comparison processor, the group rarity score of the extracted partial data with a threshold value to determine whether a singularity is present.

In a preferred embodiment, the group biometric information data measured using the sensor may include minute physical vibrations caused by respiration or a heartbeat of a subject.

In another preferred embodiment, the group biometric information data of the respiration or the heartbeat measured using the sensor may target sinusoidal amplitude.

In still another preferred embodiment, respiration data measured through the sensor may target an interval between amplitudes caused by inhalation or exhalation of the subject.

In yet another preferred embodiment, sample rarity score(i)=(CDF_vital(i)−0.5)*200 may be calculated.

In still yet another preferred embodiment, the comparing, by the comparison processor, the group rarity score with the threshold value to determine whether a singularity is present may include comparing, by the comparison processor, the group rarity score with the stored threshold value, determining, by the comparison processor, that a singularity has occurred when the group rarity score exceeds the stored threshold value in the comparing the group rarity score with the stored threshold value, and transmitting biometric information data in which the singularity has occurred to a set subject through an alarm.

In a further preferred embodiment, the calculating of the group rarity score may include calculating the group rarity score by obtaining a sum of sample rarity scores of the extracted partial data, and dividing the sum by the number of pieces of the extracted partial data.

In another aspect, the present invention provides an apparatus for determining rarity of a biometric information signal, the apparatus including a sensor configured to receive group biometric information data for a predetermined period, and a controller configured to extract partial data in the received group biometric information data, and calculate a sample rarity score of the extracted partial data and a group rarity score of the extracted partial data based on the group biometric information data, wherein the controller includes an extraction processor configured to extract partial data in the group biometric information data for the predetermined period, and a comparison processor configured to calculate a CDF based on remaining data except for the partial data in the group biometric information data, calculate a sample rarity score of the extracted partial data based on the calculated CDF, calculate a group rarity score of the extracted partial data, and compare the group rarity score with a threshold value.

In a preferred embodiment, the sensor may include minute physical vibrations caused by respiration or a heartbeat of a subject as the group biometric information data measured for the predetermined period.

In another preferred embodiment, the sensor may be configured to measure the group biometric information data of the measured respiration or heartbeat of the subject, and the controller may store sinusoidal amplitude of the measured group biometric information data.

In still another preferred embodiment, respiration data measured through the sensor may include an interval between amplitudes caused by inhalation or exhalation of the subject.

In yet another preferred embodiment, the comparison processor may calculate the sample rarity score through sample rarity score(i)=(CDF_vital(i)−0.5)*200.

In still yet another preferred embodiment, the comparison processor may compare the group rarity score with a threshold value stored in the comparison processor.

In a further preferred embodiment, when the group rarity score exceeds the stored threshold value, it may be determined that a singularity has occurred.

In another further preferred embodiment, the apparatus may further include an alarm configured to transmit biometric information data in which the singularity has occurred to the subject from the comparison processor.

In still another further preferred embodiment, the comparison processor may calculate the group rarity score by obtaining a sum of sample rarity scores of partial data selected from the extraction processor, and dividing the sum by the number of pieces of the extracted partial data.

In yet another further preferred embodiment, the extracted partial data may include at least two pieces of biometric information data that are successive in time series in the group biometric information data.

Other aspects and preferred embodiments of the invention are discussed infra.

The above and other features of the invention are discussed infra.

### BRIEF DESCRIPTION OF THE DRAWINGS

The above and other features of the present invention will now be described in detail with reference to certain exemplary embodiments thereof illustrated in the accompanying drawings which are given hereinbelow by way of illustration only, and thus are not limitative of the present invention, and wherein:

FIG. 1 illustrates a configuration diagram of an apparatus for determining rarity of a biometric information signal as an embodiment of the present invention;

FIG. 2 illustrates a flowchart of a method of determining rarity of a biometric information signal as an embodiment of the present invention; and

FIG. 3 illustrates a step of determining whether a singularity is present based on rarity of extracted partial data as an embodiment of the present invention.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Advantages and features of the present invention, and methods of achieving the advantages and the features will become clear with reference to embodiments described below in detail along with the accompanying drawings. However, the present invention is not limited to the embodiments disclosed below, and may be implemented in various different forms. Further, only this embodiment is provided to complete disclosure of the present invention and to completely inform those skilled in the art of the scope of the invention to which the present invention belongs, and the present invention is defined by the scope of the claims. Like reference numbers designate like elements throughout the specification.

Each of terms such as “ . . . section,” “ . . . unit,” and “ . . . module” described in the specification means a unit that processes at least one function or operation, which may be implemented as a combination of hardware or software, or hardware and software.

In addition, in this specification, names of components are divided into first, second, etc., to distinguish components having the same name, and the order is not necessarily limited in the following description.

In addition, various embodiments herein may be implemented as software (for example, a program) including instructions stored in a storage medium readable by a machine (for example, a computer) (machine-readable storage medium). The machine is a device capable of calling a stored instruction from the storage medium and operating according to the called command, and may include an electronic device (for example, a server) according to the disclosed embodiments. An instruction may include code generated or executed by a compiler or interpreter. The machine-readable storage medium may be provided in the form of a non-transitory storage medium. Here, “non-transitory” only means that the storage medium does not include a signal and is tangible, and does not distinguish whether data is stored semi-permanently or temporarily in the storage medium.

In addition, according to an embodiment of the present specification, methods according to various embodiments disclosed in this document may be included in a computer program product and provided. The computer program product may be traded between a seller and a buyer as a commodity. The computer program product may be distributed as a machine-readable storage medium (for example, compact disc read only memory (CD-ROM)) or online through an application store (for example, Play Store™). In the case of online distribution, at least a part of the computer program product may be temporarily stored or temporarily created in a storage medium such as a memory of a server of a manufacturer, a server of an application store, or a memory of a relay server.

The detailed description is illustrative of the present invention. In addition, the foregoing is intended to illustrate and describe preferred embodiments of the present invention, and the present invention may be used in various other combinations, modifications and environments. That is, changes or modifications are possible within the scope of the concept of the invention disclosed in this specification, within the scope equivalent to the disclosed contents, and/or within the scope of skill or knowledge in the art. The described embodiments describe the best state for implementing the technical idea of the present invention, and various changes required in specific application fields and uses of the present invention are possible. Therefore, the above detailed description of the invention is not intended to limit the invention to the disclosed embodiments. In addition, the appended claims should be construed to cover other embodiments as well.

“Singularity” of the present invention means a degree to which a significant difference occurs by comparing received data with conventional data, and the significant difference may be set differently according to a type of received biometric information data. Moreover, “singularity” may refer to biometric information data determined to be within a normal range.

The present invention relates to an apparatus for determining rarity of a biometric information signal, and provides technology for receiving group biometric information data including a plurality of pieces of biometric information during a predetermined period, and calculating rarity (rarity score) of partial biometric information data in the received data.

Here, the rarity (rarity score) is calculated based on a cumulative distribution function (CDF), and more preferably, the rarity is configured to be converted to a value of −100 to 100 based on the cumulative distribution function.

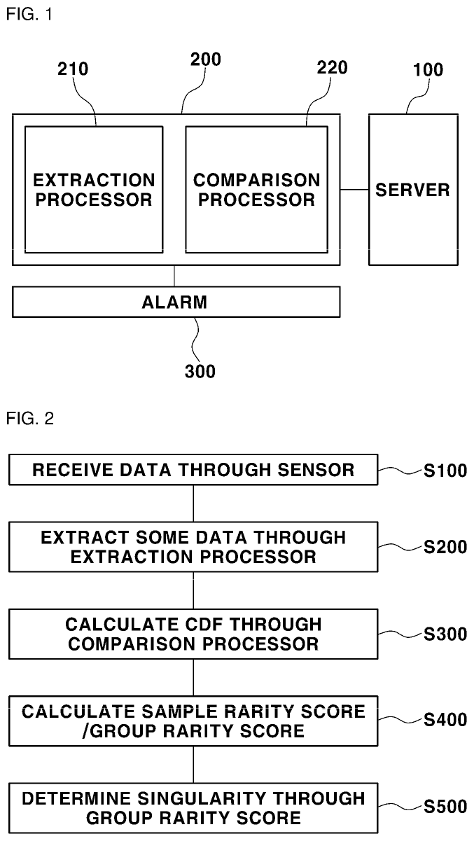

FIG. 1 illustrates a configuration diagram of an apparatus for determining rarity of a biometric information signal as an embodiment of the present invention.

As illustrated in the figure, the apparatus for determining rarity of a biometric information signal largely includes a sensor 100 configured to measure a biometric information signal, and a controller 200 configured to receive measured data from the sensor 100 and convert the received data into rarity.

The sensor 100 may detect a change in a biometric signal of a person and a change in biometric information of a person. The sensor 100 may be located in a place adjacent to a person to be measured. The sensor 100 may detect a change in a biometric signal and a change in motion of a person within a certain range based on a measurement target. For example, the place where the sensor 100 is located may include a bed, a sofa, a floor, etc. For example, the sensor 100 may be installed in a stand type or attached to a wall. One sensor 100 may be provided or a plurality of sensors 100 may be provided.

The biometric information measured by the sensor 100 may include heart rate, respiration rate, motion of a subject, a degree of distance displacement due to respiration or heartbeat, regularity of sine waves due to respiration, and regularity of heartbeat signals. Furthermore, the sensor 100 may measure regularity of sine waves caused by respiration of the measurement target during sleep. For example, the sensor 100 may determine in advance whether or not the measurement target is sleeping, and may determine sleep quality as rarity by measuring regularity of sine waves due to respiration of the target determined to be in a sleeping state. More preferably, the sensor 100 may determine whether or not the measurement target is sleeping based on motion during sleep. That is, when there is no motion of the measurement target, the controller 200 determines that the target is in a sleeping state, and then measures whether there is irregularity in respiration to determine rarity based thereon.

For example, the sensor 100 may be any one of an ultra-wideband communication (IR-UWB) sensor 100, LiDAR, frequency modulated continuous wave radar (FMCW RADAR), and Doppler radar. Ultra-wideband (UWB) communication means radio technology that uses a frequency band of 500 MHz or more or has a value defined as a specific bandwidth of 25% or more. The specific bandwidth refers to a bandwidth of a signal relative to a center frequency. UWB communication is radio technology that uses a broadband frequency and has various advantages such as high distance resolution, transparency, strong immunity to narrowband noise, and coexistence with other machines that share a frequency. For example, UWB communication has the advantage of being able to detect even minute motions of an object at an ultra-precise distance resolution of 1 cm or less.

Impulse-radio ultra-wideband radar (hereinafter referred to as UWB radar) technology is a system in which such UWB communication technology is combined with radar, and refers to radar technology that transmits an impulse signal of a significantly short duration having broadband characteristics in the frequency domain, receives a signal reflected from an object or a person, and recognizes a surrounding situation. In a UWB radar system, a signal generator generates an impulse signal having a time width of several nanoseconds to several picoseconds and radiates the signal at a wide angle or an angle of a narrow band through a transmission antenna. The radiated signal is reflected by various objects or people in the environment, and the reflected signal may be converted into a digital signal through a reception antenna and an analog-to-digital converter (ADC).

LiDAR may emit a laser pulse, receive the laser pulse reflected by a neighboring target object to return, and measure a distance from the LiDAR to the object. The LiDAR may detect a speed of the target object, a shape of the target object, etc. in addition to the distance to the target object, and furthermore may be used to generate a 3D image of the surroundings.

A biometric signal measured through the sensor 100 including radar or LiDAR may include at least one of a heart rate or respiration of a person. Preferably, the biometric signal may be the heart rate of the person. The sensor 100 may detect motion of a chest or a stomach of the person by receiving a signal reflected by the person, and detect the heart rate or the respiration of the person based thereon.

The sensor 100 may detect a shortest distance to a person. The sensor 100 may detect the shortest distance to the person based on a specific location of the person. For example, the specific location may mean a part of a body of the person, such as a head, a torso, or a leg, and the specific location may mean a location from which the shortest distance from the sensor 100 to the person is derived. Accordingly, the specific location may refer to a location that is changed in real time according to motion of the person. For example, the sensor 100 may measure a chest gap due to inhalation or exhalation of a breathing subject, and may measure a distance between amplitudes.

The controller 200 may receive biometric information data measured through the sensor 100 in real time. Alternatively, the controller 200 may receive biometric information data received during a set period. The controller 200 may set a predetermined period to receive a plurality of pieces of biometric information data.

As an embodiment of the present invention, data measured for 14 days as a predetermined period for measuring group biometric information data may be included, and extracted partial data may include most recently measured data in units of 12 hours, which is group biometric information data, and when rarity of the extracted partial data is obtained using a CDF obtained based on remaining data except for partial data extracted from the data for 14 days, “a rarity score of extracted partial data” is calculated as a group rarity score.

Here, several numerical values actually measured throughout the day are set as extracted partial data, and specificity for each piece of extracted partial data is calculated as a sample rarity score. That is, since each measured value is a numerical value in a short time, an individual value may vary. Therefore, when an alarm is set depending thereon, excessively frequent transmission of an alarm 300 may occur. In order to prevent this, comparison with a threshold value is performed using a group rarity score of extracted partial data.

In other words, as data generated as an event exceeding a threshold value, depending on the situation, a heart rate may increase in a short time, and a respiratory rate may increase immediately after extreme physical activity. Thus, when singularity is determined depending on data measured in a short time, analysis meaning of the biometric information signal may be halved. Therefore, the present invention may set one day or 12 hours as a period of extracted partial data, and analyzing specificity for a certain set period is more consistent with analysis of biometric information of a subject than comparing a measured value in a short time with a threshold value. In summary, the extracted partial data is values belonging to a certain interval, individual rarity may be expressed as a sample rarity score, and total rarity for the entire group of the extracted partial data as the values belonging to the certain interval may be expressed as a group rarity score.

The controller 200 may include an extraction processor 210 and a comparison processor 220. The extraction processor 210 may extract partial data from a plurality of pieces of biometric information data measured during a predetermined period. Here, the extracted partial data means data for determining whether or not singularity is recognized among the plurality of pieces of biometric information data measured for the predetermined period.

The extraction processor 210 performs data extraction to include at least two pieces of data that are successive in time series. In addition, the extraction processor 210 may be configured to select partial data extracted by performing extraction at least once in the group biometric information data.

The comparison processor 220 may calculate a sample rarity score based on partial data extracted from the extraction processor 210. As for the sample rarity score, a cumulative distribution function is calculated based on remaining biometric information data excluding partial data extracted through the extraction processor 210 from the group biometric information data measured by the sensor 100. The sample rarity score for extracted partial biometric information data is calculated based on the cumulative distribution function calculated in this way. More preferably, the sample rarity score is calculated as in the following Equation 1.

$$( i - thSampleRarityScore ) = ( C D {F}_{vital ( i )} - 0.5 ) \times 200 \qquad [ Equation 1 ]$$

Here, viral(i) denotes an ith piece of biometric information data for determining whether a singularity is present among a plurality of pieces of biometric information data.

The comparison processor 220 converts the sample rarity score so that the sample rarity score has a value of −100 to 100 through the above Equation 1.

In addition, the comparison processor 220 calculates a group rarity score through the sample rarity score. The group rarity score is calculated by dividing the sum of sample rarity scores by the number of pieces of extracted partial data. That is, the comparison processor 220 calculates an average rarity score of a specific group as extracted partial data. The calculated group rarity score is compared with a threshold value stored in the controller 200 to determine whether a singularity has occurred in a specific group. More preferably, the comparison processor 220 compares the threshold value and the group rarity score, and determines that a singularity has occurred upon determining that the group rarity score exceeds the threshold value.

Furthermore, the controller 200 is configured to transmit corresponding biometric information data through the alarm 300 upon determining that the group rarity score exceeds the threshold value. More preferably, the alarm 300 may transmit information of the corresponding biometric information data to a target set through a server, and may include a hospital, a medical institution, etc. as a target.

FIG. 2 illustrates a flowchart of a method of determining rarity of a biometric information signal as an embodiment of the present invention.

The controller 200 receives a plurality of pieces of biometric information data during a predetermined period and stores the group biometric information data in the controller 200 (S100). The stored biometric information data is stored in the controller 200 by sequentially labeling each piece of data (i=n) from a time of measurement (i=1). For example, the group biometric information data includes biometric information data measured N times.

Thereafter, the extraction processor 210 performs a step of extracting partial data for determining a singularity in the group biometric information data stored in the controller 200 (S200). The partial data extracted here may include at least one piece of data, and in an embodiment of the present invention, 10 pieces of latest measured data among 100 pieces of group biometric information data are extracted as targets. More preferably, the extraction processor 210 is configured to include at least two pieces of biometric information data consecutive in time series in the group biometric information data measured N times as the extracted partial data. In addition, the extraction processor 210 may include the number of times that the partial data in the group biometric information data is extracted at least once. Therefore, the comparison processor 220 determines rarity for the partial data extracted one or more times.

The biometric information data to be measured is configured to measure minute vibrations caused by respiration or heartbeat of the subject through the sensor 100. Furthermore, group biometric information data of measured respiration or heartbeat may measure sinusoidal amplitude according to vibration. In addition, respiration data may measure intervals between amplitudes of vibrations caused by inhalation or exhalation of the subject.

The comparison processor 220 calculates a cumulative distribution function based on remaining data except for partial data in the group biometric information data (S300), and calculates a sample rarity score of the extracted partial data based on the calculated cumulative distribution function (S400). The sample rarity score calculates rarity through (i-thSampleRarityScore)=(CDFᵥᵢₜₐₗ₍ᵢ₎−0.5)×200 based on each piece of measurement data. Moreover, the comparison processor 220 calculates a group rarity score through the calculated sample rarity score. The group rarity score is calculated by obtaining a sum of sample rarity scores of the extracted partial data and dividing the sum of the sample rarity scores by the number of pieces of the extracted partial data (S400).

The comparison processor 220 performs a step of comparing the calculated group rarity score with a threshold value stored in the controller 200. The comparison processor 220 is configured to determine that a singularity has occurred (S500) when the group rarity score exceeds the threshold value, and to transmit biometric information data in which the singularity has occurred to a set target through the alarm 300.

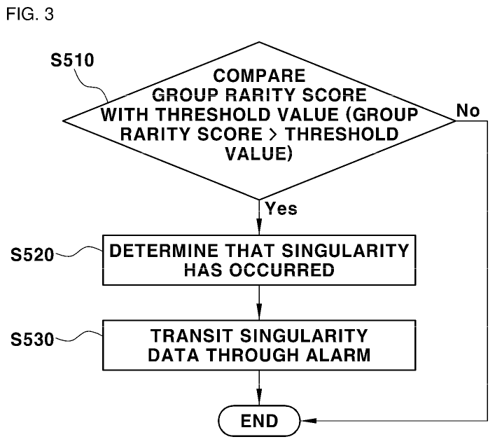

FIG. 3 illustrates a step of performing determination by comparing the calculated group rarity score with the threshold value stored in the controller 200.

As illustrated in the figure, the comparison processor 220 performs a step of comparing the calculated group rarity score with the threshold value stored in the controller 200 (S510). When the calculated group rarity score exceeds the threshold value stored in the controller 200 (S510), determination is made that a singularity has occurred based on biometric information data measured for a predetermined period (S520).

The singularity is determined based on biometric information data measured for a predetermined period, and thus may be interpreted as a significant change being specified. Furthermore, a measurement time of the biometric information data at which the singularity is determined and a biometric information signal measured at the corresponding time are stored in the controller 200 and transmitted through the alarm 300. The controller 200 may be configured to set a transmission object through the alarm 300, and transmit data and a time of the biometric information signal at which the singularity is generated to the set object (S530).

Alarms that are sent out may include first to fourth alarms according to a degree of exceeding the threshold value. For example, the controller 200 transmits a first alarm, a second alarm, a third alarm, and a fourth alarm to a terminal using a wireless communication method.

Moreover, at least one alarm may be transmitted according to a type of each biometric information signal. For example, signals according to respiration and heartbeat may be transmitted simultaneously or separately.

The present invention may obtain the following effects according to a combination and usage relationship of the present embodiment and configurations described above.

The present invention determines rarity of the partial data extracted based on group biometric information data measured during a predetermined period, and provides an effect of being able to determine a singularity through a probability relationship between other data measured during the period and the extracted data.

The present invention performs determination by comparing extracted data corresponding to a certain area or time among pieces of data measured for a certain period with data measured for the certain period, and has an effect of being able to analyze a singularity of a coherent biometric information signal of data for the certain period rather than analyzing each piece of data.

The above detailed description is illustrative of the present invention. In addition, the foregoing illustrates and describes preferred embodiments of the present invention, and the present invention may be used in various other combinations, modifications and environments. That is, changes or modifications are possible within the scope of the concept of the invention disclosed in this specification, within the scope equivalent to the disclosed contents, and/or within the scope of skill or knowledge in the art. The described embodiment describes the best state for implementing the technical idea of the present invention, and various changes required in specific application fields and uses of the present invention are possible. Therefore, the above detailed description of the invention is not intended to limit the invention to the disclosed embodiments. In addition, the appended claims should be construed to cover other embodiments as well.

What is claimed is:

1. A method of determining rarity of a biometric information signal, the method comprising:
receiving group biometric information data for a certain period through a sensor;
extracting, by an extraction processor, partial data in the received group biometric information data;
calculating, by a comparison processor, a cumulative distribution function (CDF) based on remaining data except for the partial data in the group biometric information data;
calculating, by the comparison processor, a sample rarity score of the extracted partial data based on the calculated CDF, and calculating a group rarity score from the sample rarity score; and
comparing, by the comparison processor, the group rarity score of the extracted partial data with a threshold value to determine whether a singularity is present.

2. The method of claim 1, wherein the group biometric information data measured using the sensor includes minute physical vibrations caused by respiration or a heartbeat of a subject.

3. The method of claim 2, wherein the group biometric information data of the respiration or the heartbeat measured using the sensor targets sinusoidal amplitude.

4. The method of claim 2, wherein respiration data measured through the sensor targets an interval between amplitudes caused by inhalation or exhalation of the subject.

5. The method of claim 1, wherein sample rarity score(i)=(CDF_vital(i)−0.5)*200 is calculated.

6. The method of claim 1, wherein the comparing, by the comparison processor, the group rarity score with the threshold value to determine whether a singularity is present comprises:
comparing, by the comparison processor, the group rarity score with the stored threshold value;
determining, by the comparison processor, that a singularity has occurred when the group rarity score exceeds the stored threshold value in the comparing the group rarity score with the stored threshold value; and
transmitting biometric information data in which the singularity has occurred to a set subject through an alarm.

7. The method of claim 1, wherein the calculating of the group rarity score comprises calculating the group rarity score by obtaining a sum of sample rarity scores of the extracted partial data, and dividing the sum by the number of pieces of the extracted partial data.

8. An apparatus for determining rarity of a biometric information signal, the apparatus comprising:
a sensor configured to receive group biometric information data for a predetermined period; and
a controller configured to extract partial data in the received group biometric information data, and calculate a sample rarity score of the extracted partial data and a group rarity score of the extracted partial data based on the group biometric information data,
wherein the controller comprises:
an extraction processor configured to extract partial data in the group biometric information data for the predetermined period; and
a comparison processor configured to calculate a CDF based on remaining data except for the partial data in the group biometric information data, calculate a sample rarity score of the extracted partial data based on the calculated CDF, calculate a group rarity score of the extracted partial data, and compare the group rarity score with a threshold value.

9. The apparatus of claim 8, wherein the sensor includes minute physical vibrations caused by respiration or a heartbeat of a subject as the group biometric information data measured for the predetermined period.

10. The apparatus of claim 8, wherein the sensor is configured to measure the group biometric information data of the measured respiration or heartbeat of the subject, and the controller stores sinusoidal amplitude of the measured group biometric information data.

11. The apparatus of claim 10, wherein respiration data measured through the sensor includes an interval between amplitudes caused by inhalation or exhalation of the subject.

12. The apparatus of claim 8, wherein the comparison processor calculates the sample rarity score through sample rarity score(i)=(CDF_vital(i)−0.5)*200.

13. The apparatus of claim 8, wherein the comparison processor compares the group rarity score with a threshold value stored in the comparison processor.

14. The apparatus of claim 13, wherein, when the group rarity score exceeds the stored threshold value, determination is made that a singularity has occurred.

15. The apparatus of claim 14, further comprising an alarm configured to transmit biometric information data in which the singularity has occurred to the subject from the comparison processor.

16. The apparatus of claim 8, wherein the comparison processor calculates the group rarity score by obtaining a sum of sample rarity scores of partial data selected from the extraction processor, and dividing the sum by the number of pieces of the extracted partial data.

17. The apparatus of claim 8, wherein the extracted partial data includes at least two pieces of biometric information data that are successive in time series in the group biometric information data.
